(19) United States
(12) Patent Application Publication
(10) Pub. No.: US 2010/0241060 A1
(43) Pub. Date: Sep. 23, 2010

(54) SURGICAL DEVICES AND METHODS

(76) Inventors: Keith ROIZMAN, Los Angeles, CA (US); H. Michael Lambert, Austin, TX (US); Patrick Michael Elliott, Upland, CA (US)

Correspondence Address: OSHA LIANG L.L.P., TWO HOUSTON CENTER, 909 FANNIN, SUITE 3500, HOUSTON, TX, 77010, US

(21) Appl. No.: 12/406,292

(22) Filed: Mar. 18, 2009

(51) Int. Cl. A61F 9/007; A61M 5/00; A61F 2/14

(52) U.S. Cl. 604/22; 604/132; 604/257; 604/264; 623/5.11

(57) ABSTRACT

Surgical devices and methods are described. The devices are for implanting fluidic material in a patient's eye and comprise a nozzle and a housing having a slide and a bellows. The bellows is adapted to undergo compression or decompression. The surgical procedures make use of the surgical devices and include a tissue translocation surgical procedure, a retinal implantation surgical procedure, and a corneal transplantation surgical procedure.

### FIELD

The present disclosure relates to surgical devices and methods. In particular, it relates to surgical devices and methods to implant and/or translocate fluidic material in a patient's eye and to remove undesirable materials such as debris, dead cells, blood, etc., from the patient's eye.

### BACKGROUND

In vitreo-retinal surgery, simple instruments and devices, such as subretinal forceps, light probes, and other devices based on a plunger+tube/barrel injector, have been used on a limited basis. There is a need for novel surgical technologies with the capability to deliver and remove materials in the subretinal space. A further need is that of providing for safe delivery and expression of corneal endothelial cell layers into the anterior segment of the eye.

### SUMMARY

According to a first aspect, a device adapted to implant fluidic material in a patient's eye is provided, comprising: a nozzle; and a housing, connected with the nozzle, the housing comprising a slide and a bellows, the bellows located between the nozzle and the slide, wherein: the slide is adapted to slide towards the nozzle or away from the nozzle; the bellows is adapted to undergo compression or decompression, sliding of the slide towards the nozzle occurs together with the compression of the bellows; and sliding of the slide away from the nozzle occurs together with the decompression of the bellows.

According to a second aspect, a device adapted to implant fluidic material in a patient's eye is provided, comprising: a housing, adapted to be connected with a nozzle, the housing comprising a slide and a bellows, the bellows to be located between the nozzle and the slide, wherein: the slide is adapted to slide towards a distal end of the housing or away from the distal end of the housing; the bellows is adapted to undergo compression or decompression, sliding of the slide towards the distal end of the housing occurs together with the compression of the bellows; and sliding of the slide away from the distal end of the housing occurs together with the decompression of the bellows.

According to a third aspect, a nozzle for intracorneal implantation surgical procedures is provided, comprising: a body region; a tip region; a holding region between the body region and the tip region, the holding region defining a channel adapted to be filled with fluid and contain corneal cell layers for implantation; and a lens glide located after the tip region.

According to a fourth aspect, a nozzle for surgical procedures is provided, the nozzle adapted to be connected to a surgical procedure preparation device, the nozzle comprising: a lumen, to allow vision of operation of the surgical procedure preparation device on a patient's body, and a nozzle tip, the nozzle tip comprising i) a first opening into which material is adapted to be suctioned from the patient's body or from which material is adapted to be injected into the patient's body, and ii) a second opening where a distal end of the lumen is located.

According to a fifth aspect, a sealing arrangement is provided, comprising: a nozzle; an adaptor connected with the nozzle, the adaptor being for connecting the nozzle with a body of a surgical device; and a cover, wherein the cover: i) surrounds the nozzle, ii) contacts the adaptor through a snap-fit connection, and iii) contacts the body of the surgical device.

According to a sixth aspect, a cartridge for medical use is provided, comprising: a nozzle; a cover surrounding the nozzle, the cover and the nozzle defining an internal chamber where storage and/or preservation media are adapted to be located; and a cap adapted to lock the cover and the nozzle inside the cover.

According to a seventh aspect, a surgical translocation procedure for operating on patients with macular degeneration is provided, comprising: performing vitrectomy on the patient; removing undesired material from a subretinal area of the patient; obtaining tissue from the subretinal area of the patient; and translocating the tissue from the subretinal area of the patient to the submacular area of the patient.

According to an eighth aspect, a surgical implantation procedure for operating on patients with macular degeneration is provided, comprising: performing vitrectomy on the patient; removing undesired material from a subretinal area of the patient; and implanting tissue in a submacular area of the patient.

According to a ninth aspect, a surgical corneal transplantation procedure is provided, comprising: performing a first incision in the corneal region of a patient; performing a further incision adapted to create space for a forceps to be used during the procedure; inserting a corneal transplantation device into the first incision, the corneal transplantation device comprising corneal transplantation tissue; inserting a forceps into the further incision; expressing the corneal transplantation tissue from the corneal transplantation device; and manipulating the corneal transplantation tissue with the forceps.

### BRIEF DESCRIPTION OF THE DRAWINGS

FIG. 1 shows an exploded view of a first embodiment of a surgical device.

FIG. 2 shows a perspective view of the first embodiment.

FIG. 3 shows a top view of a locking arrangement in accordance with the disclosure.

FIG. 4 shows a conceptual view explaining the operation of the locking arrangement of FIG. 3.

FIGS. 5A-5D show views of a nozzle to be used in intracorneal surgery.

FIGS. 6, 6A and 6B show cross sectional views of further embodiments of the surgical device.

FIGS. 7A and 7B show a perspective view and a sectional view of a nozzle for a translocation device, where a stop portion is also shown.

FIGS. 7C and 7D show a perspective view and a sectional view of a nozzle for an implantation device.

FIGS. 7E and 7F show a perspective view and a sectional view of an intracorneal nozzle.

FIG. 8 shows a nozzle with a snake-head tip.

FIG. 9A shows a sectional view of a nozzle for a preparation device.

FIG. 9B shows a partial perspective view of the distal end of the nozzle of FIG. 9A.

FIG. 9C shows a further embodiment of a nozzle for a preparation device.

FIGS. 10, 10A and 10B show a nozzle cover acting as a cartridge, in accordance with a further embodiment of the present disclosure.

FIGS. 11 and 12 show a further embodiment of a nozzle cartridge.

FIG. 13 shows steps of a translocation surgical procedure in accordance with the disclosure.

FIG. 14 shows steps of an implantation surgical procedure in accordance with the disclosure.

FIG. 15 shows steps of a corneal transplantation surgical procedure in accordance with the disclosure.

### DETAILED DESCRIPTION

A first embodiment of the present disclosure relates to a subretinal implantation device. The subretinal implantation device is suited to implant fluidic material into the subretinal region of a patient's eye by way of fluidic motion.

According to some embodiments of this disclosure, such fluidic material can comprise tissue, intact organized cell layers, biologic agents, bioactive agents, and any other organic or inorganic matter that can be used by a surgeon in retinal surgery. The fluidic material can also comprise, for example, one or more of: a sterile balance saline solution, Optisol®, similar fluids for intraocular use, storage and preservation media, retinal tissue, growth factors and/or other bioactive agents, autologous cells, fetal cells, stem cells, derived intact retinal cell layers, and intact corneal layers. Given its small dimensions and its use in eye surgery (e.g., retinal, corneal etc.) operations, such material can also be defined as a microfluidic material.

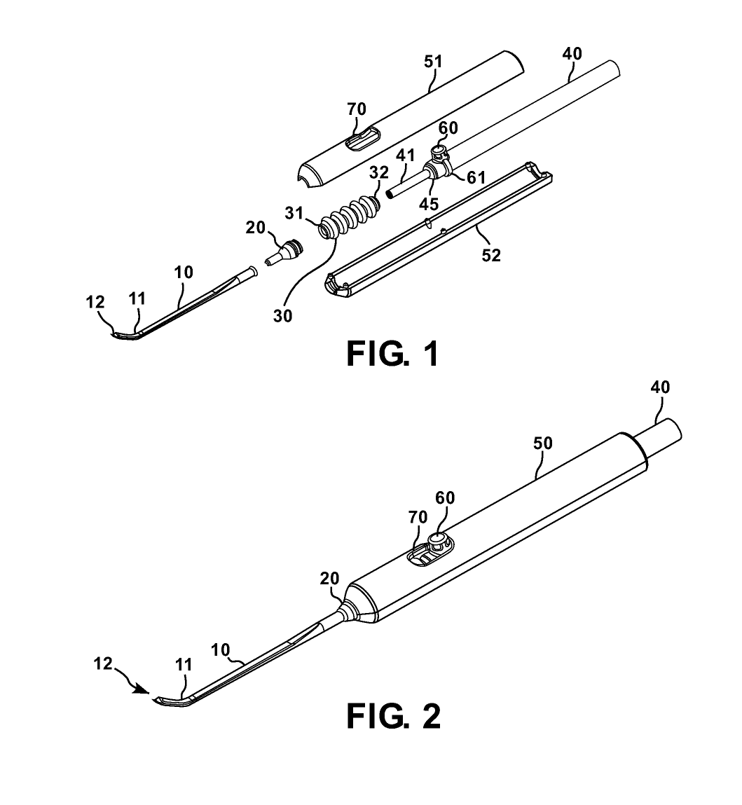

As shown in the exploded view of FIG. 1 and the perspective view of FIG. 2, the implantation device comprises a nozzle (10), a nose piece or adaptor (20), a bellows (30), a slide (40), and a housing (50) comprising a first housing portion (51) and a second housing portion (52).

As shown in FIG. 1, the adaptor (20) connects the nozzle (10) with the bellows (30) along a first end (31) of the bellows (30). A second end (32) of the bellows (30) is connected to the slide (40). The first and second housing portions (51), (52) encapsulate the bellows (30) and the slide (40). The two housing portions can be, for example, clam shell components joined along the longitudinal axis of the housing.

As shown in FIG. 2, portions of the nose piece (20) and the slide (40) can protrude out of the housing (50). The protruding part of the slide can be covered by a protective cap (not shown), if desired.

FIGS. 1 and 2 also show a locking arrangement (60), (70). In the example shown in these figures, the locking arrangement (60), (70) is a bayonet-like lock that can assume a blocking condition where movement of the slide (40) is blocked and a sliding condition where movement of the slide (40) is allowed.

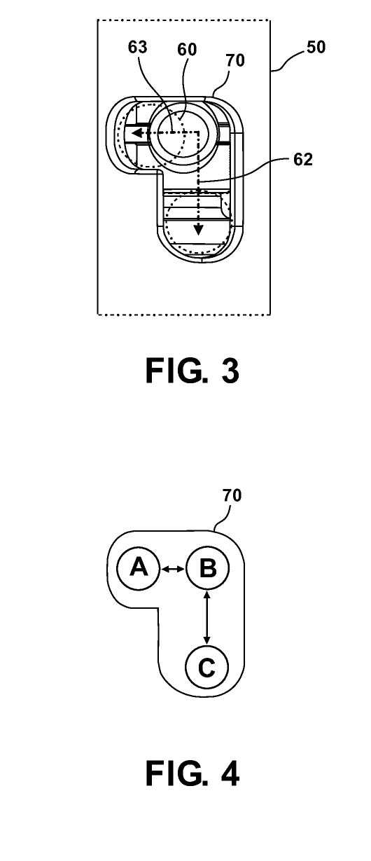

The locking arrangement (60), (70) will now be described with some additional detail with reference to FIG. 3, where an enlarged top view is shown. In particular, the locking arrangement comprises an engagement member (60) and an L-shaped cut-out portion (70) housing the engagement member (60). As also shown in FIG. 2, the engagement member (60) is located on top of the slide (40) and can be made integral with the slide (40) through provision, for example, of a ring (61) encircling the slide (40). Therefore, rotation of the engagement member (60) causes rotation of the slide (40).

With continued reference to FIG. 3, when the engagement member (60) is located in the top right area of the L-shaped cut-out portion (70), the engagement member (60), and the slide (40) with it, is allowed to slide forward and/or backwards along direction (62). On the other hand, when movement of the engagement member (60) along direction (63) brings the engagement member (60) in the top left area of the L-shaped cut-out portion (70), a locked condition is reached, because the limited longitudinal dimension of that area prevents longitudinal movement of the engagement member (60).

During operation of the implantation device, fluidic material is located in the nozzle (10) and is adapted to be implanted subretinally by being expelled from the nozzle (10) through pressure exercised by the bellows (30). The bellows (30) exercises a pressure inside the nozzle (10) through movement of the slide (40) toward the nozzle (10). In particular, such movement will compress the bellows (30) and such compression will generate, in turn, a hydraulic pressure inside the nozzle area, which will expel the fluidic material out of the nozzle (10) with precise, controlled motion as determined by the operating surgeon. According to an embodiment of the present disclosure, the implantation device is capable of generating 1 to 1.25 psi of positive pressure in the nozzle area. Pressure duration is a function of the compression stroke of the bellows associated to the longitudinal extension of the L-shaped cut-out portion (70) along direction (62) shown in FIG. 3.

As schematically illustrated in FIG. 4, before operation, the engagement member (60) is located in position A of the L-shaped cut-out portion (70). In order to perform implantation, the surgeon will initially move the engagement member (60) from position A to position B, thus disengaging the lock. After that, the surgeon will move the engagement member (60) longitudinally towards position C, thus compressing the bellows (30) and exercising a pressure inside the nozzle (10) that will expel the material from the nozzle (10). Therefore, the forward motion of engagement member (60) pushes the slide (40).

Reference will now be made to the nozzle (10). According to one example of the implantation embodiment of the present disclosure, the nozzle (10) can have a bevel region (11) in proximity of its distal end (12), as shown in FIGS. 1 and 2. The function of the bevel is that of forming a cutting edge for the surgeon. In particular, the bevel region (11) can have an inclination of about 45 degrees to about 60 degrees. Such inclination allows for easier entry into a surgeon-made retinotomy (retinal incision), so that the nozzle tip (12) can enter the subretinal space and the device can perform its required function.

As shown in the embodiment discussed so far, nozzle pressure is exercised by way of a bellows (30) instead of a plunger plus tube/barrel arrangement. As already discussed with reference to FIG. 1, the bellows (30) has a first end (31) connected with the adaptor (20) and a second end (32) connected with section (45) of the slide (40). Such connection persists at all times, both during a compressed condition of the bellows (30) and during a non-compressed condition of the bellows (30). Such connection provides an improvement over a plunger plus tube/barrel arrangement, because there is no relative displacement and friction of one element with respect to the other. In particular, compression and decompression of bellows (30) will occur at the same time of forward and backward movement of the slide (40), respectively, without relative movement of the end (32) of bellows (30) with respect to the slide (40). Further, a mechanical touching of such fragile, intact cell layers/materials by a plunger can damage, crush, and “knock off” important cells attached to the intact cell layers/materials.

Moreover, in a plunger and tube/barrel arrangement, an initial higher force and pressure has to be exercised to overcome the initial and ongoing friction that typically occurs in a plunger and tube/barrel arrangement in order to initiate and maintain a fluid flow. In very delicate microsurgical procedures like those involved in subretinal surgery, this initial force creates an initial jolt and ongoing undesirable motion which can create a risk of a damaging, rapid, intense pressure expression, as well as damaging movement of the nozzle in the subretinal space, leading to potentially significant and permanent damage to both the patient's eye and the fragile intact cell layer implant. Such problem is overcome by the bellows according to the present disclosure because, with such bellows, the degree of control is much higher. In particular, in accordance with what is shown in FIGS. 1-4, the bellows (30) provides a minimal and uniform force from the beginning to the end of the stroke length, together with complete fluidic material expression. Additionally, the force applied to engagement element (60) is a linear force, not affected by a natural tendency to “clamp down” and cause undesirable movement or rotation of the nozzle while in the subretinal space prior to the expression, thus causing the aforementioned damage to the patient's eye and fragile intact cell layers that would occur while gripping a plunger and tube/barrel arrangement. In other words, in accordance with the present disclosure, the linear movement of the engagement element (60) that activates the slide (40) is separated by the gripping of the device and more precisely controllable, thereby essentially eliminating any undesirable motion or rotation of the nozzle during material expression in the subretinal space.

A further aspect of the embodiment shown in FIG. 1 is that a forward portion (41) can be provided. The forward portion (41) is attached to the slide (40) and located inside the bellows (30). Location of the forward portion (41) inside the bellows (30) is advantageous, because it prevents the bellows (30) from collapsing.

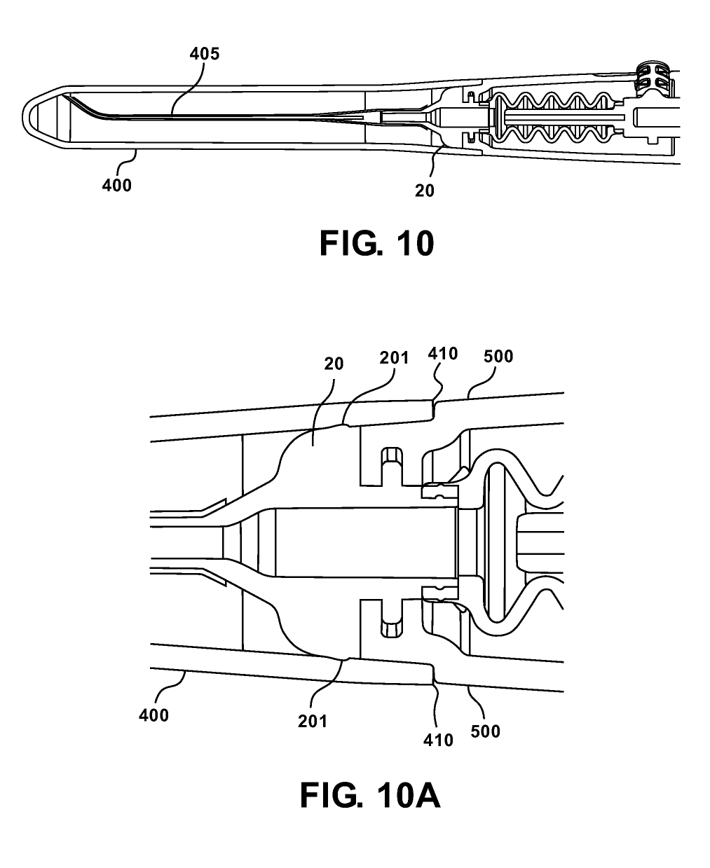

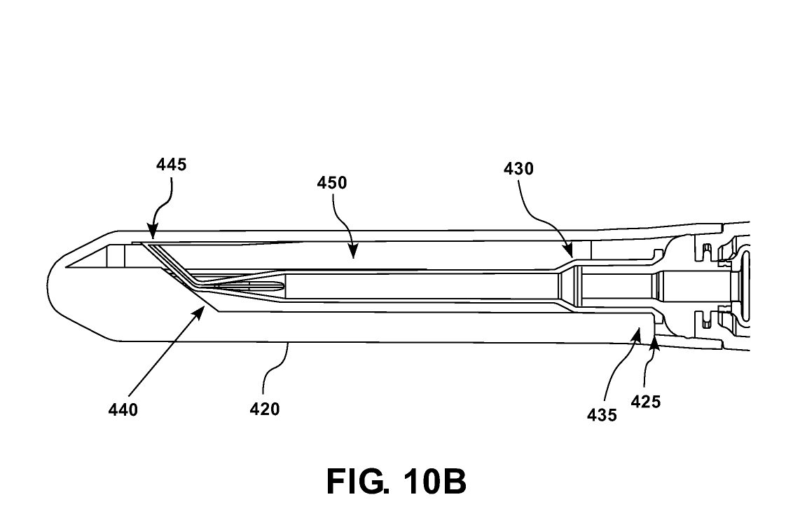

Location of the fluidic material adapted to be used with the device according to the present disclosure will now be discussed. According to an embodiment of the present disclosure, the microfluidic material can be located in the nozzle. According to a further embodiment, the microfluidic material can be located in the bellows. According to a still further embodiment, the microfluidic material can be located both in the bellows and the nozzle. A further location of the microfluidic material will be described with reference to the cap canister embodiment shown in FIG. 10 of the present application.

Given its small dimensions, the bellows will be sometimes defined, throughout the present disclosure, as a microbellows. Location of the microfluidic material in the microbellows region provides the bellows with an additional feature in addition to the springing, forward/backward, positive/negative pressure and expression/suction features described above.

A second embodiment of the present disclosure relates to an intracorneal implantation device, the structure of which is similar to the device shown in FIGS. 1-4 but for the shape of the nozzle. In particular, the nozzle of the intracorneal implantation device according to the present disclosure is shaped to allow implantation of intact corneal cell layers into the anterior chamber of the eye.

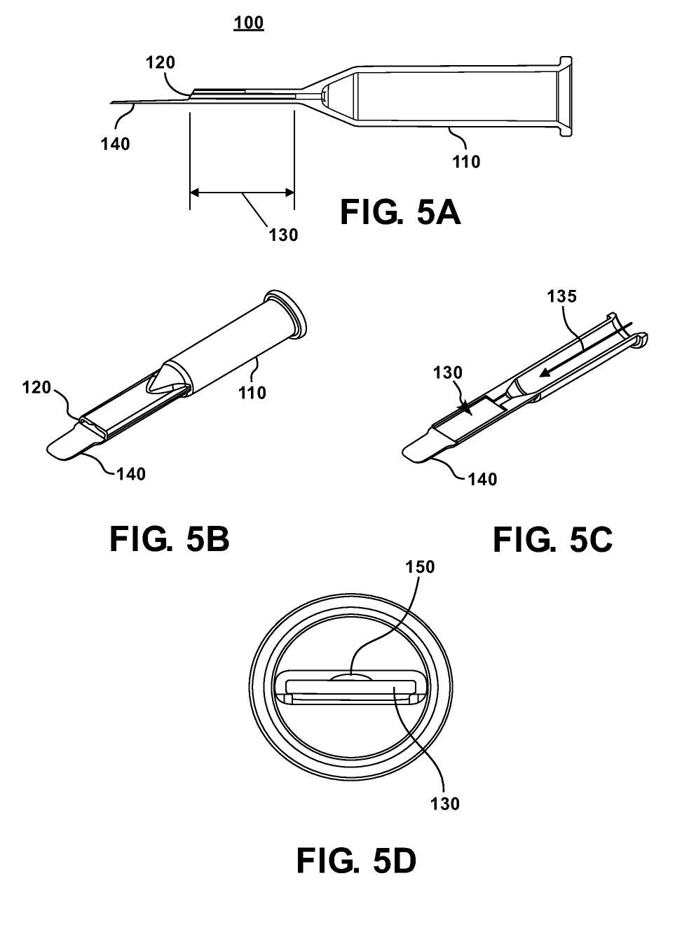

FIGS. 5A-5D show four views of a nozzle adapted to be used with such intracorneal implantation device. More in particular, FIG. 5A shows a side cross sectional view of the nozzle, FIG. 5B shows a perspective view of the nozzle, FIG. 5C shows a partial perspective view of the nozzle, and FIG. 5D shows a partial front view of the nozzle. As shown in FIGS. 5A-5D, nozzle (100) has a body region (110) and a tip region (120). A holding region or channel (130) is located between the body region (110) and the tip region (120). The holding region (130) is a hollow region adapted to be filled with fluid and contain the corneal cell layers for implantation. See, for example, FIG. 5C, where the holding region (130) and the fluid path (135) are shown.

A lens glide (140) can be provided immediately after the tip region (120). The lens glide (140) will provide a platform for the insertion of the cell layers in order to center the eye to protect the lens and the pupil, similarly to what happens with anterior chamber lenses insertion.

The tip region (120) can be tapered, as shown in FIG. 5A, in order to facilitate insertion by allowing the “nose” of the hollow holding region (130) to be partially inserted into the corneal incision. Further, as shown in FIG. 5D, a groove or notch (150) can be located, according to an embodiment of the present disclosure, in the upper center of holding region (130). The presence of such groove allows an edge of the tissue inside holding region (130) to be grasped by a forceps. In particular, an incision can be made on the opposite side of the cornea and the forceps inserted and slipped across the anterior chamber over the glide to facilitate tissue entry into the anterior segment while unfolding and exacting central placement of the intact cell layer/tissue.

Reference will now be made to a third embodiment of the present disclosure, where a subretinal translocation device will be shown. The subretinal translocation device according to the present disclosure is suitable not only to translocate and implant material such as tissue and/or intact cell layers to a subretinal region of a patient's eye, but also to initially take such material from a location in the patient's eye and relocate it to another location. According to the latter aspect of such embodiment, the subretinal translocation device operates an autologous transplant, i.e. the tissue and/or intact cell layers are taken from a location inside the eye of the same patient to whom the cells are to be later implanted. Therefore, according to this third embodiment, the subretinal translocation device is provided with a suctioning ability, in order to allow tissue or intact cell layer intake.

The implantation device already shown in FIGS. 1-4 can also be used as a translocation device in accordance with the teachings provided below. In particular, the slide (40) is configured to be hollow in order to establish a fluid path.

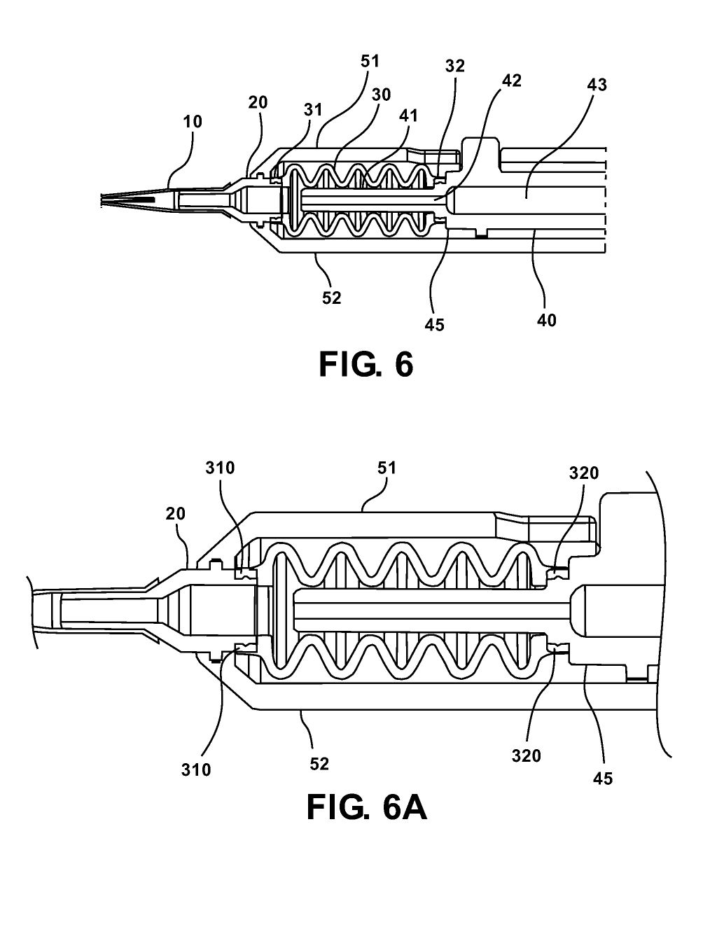

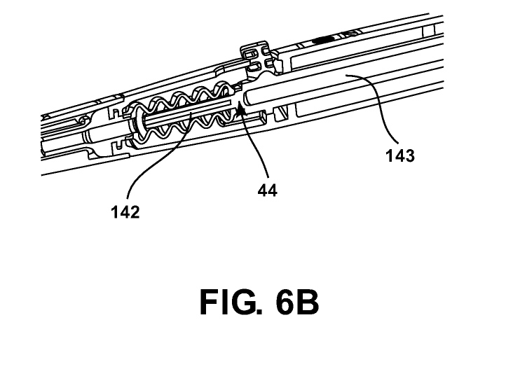

As better shown in the partial cross-sectional view of FIG. 6, the hollow sections (42), (43) of the slide (40), the inside of the bellows (30), the inside of the nose piece (20) and the inside of the nozzle (10) form a fluidic path that allows negative pressure to be formed through movement of the slide (40) away from the nozzle (10) or, alternatively, through suction of air or fluid from the distal end of the slide (40) and beyond the slide away from the nozzle (10). In this way, tissues or intact cell layers located outside the device and proximate to the nozzle (10) can be suctioned into the nozzle (10) during surgery and retained by the device. The hollow section (42) and the hollow section (43) can have different diameters, as shown in FIG. 6. This will allow pressure reduction and/or control when creating vacuum through suctioning. As to the outer diameter of section (41), it should be noted that such diameter acts to prevent collapse of the bellows (30) upon compression, as already noted with reference to an example of the implantation device.

In accordance with what was stated in the previous paragraph, two suctioning or capturing arrangements can be provided for the translocation device. In a first arrangement, suction occurs through movement of the engagement lock (60) from position C to position B, see also FIG. 4, thus providing a bellows-generated negative pressure. Therefore, differently from the implantation device embodiment, in the translocation embodiment, the initial condition of the device is with the engagement member (60) in position C and with the bellows (30) in a compressed condition.

According to a second arrangement, when the suctioning pressure to be exercised by movement of the locking arrangement from position C to position B is not enough, the device can be associated with a vacuum generator and a foot pedal or other external vacuum means, to control suctioning through the distal end of the slide (40). To this purpose, a Luer® connection can be provided, for connection purposes. For example, an adapter can be provided together with the device. The adapter comprises a female Luer® lock on its proximal end and a male Luer® on its distal end, with a cylindrical extension for insertion into the recess at the proximal end of the hand piece of the device. After connection, the female Luer® lock would be protruding for connection to a suction device.

It should be noted that suctioning in accordance with the present disclosure also allows for continuous removal of debris and other undesirable materials gently from the patient's eye and subretinal space. This function will be explained in additional detail when addressing a preparation device in accordance with the present disclosure, as later discussed.

As also shown in FIG. 6 and similarly to the implantation embodiment, the bellows (30) is integrally connected with adaptor (20) along its first end (31) and integrally connected with portion (45) of slide (40) along its second end (32). The person skilled in the art will note that, in the translocation embodiment, such integral connections allow the bellows (30) to operate as a seal of the fluid path during suctioning.

FIG. 6A is an enlarged view of FIG. 6, and will be now discussed to explain the connections of the bellows (30) to adaptor (20) and slide (40) in additional detail. In particular, first end (31) comprises a flat circular protruding region (310), and second end (32) comprises a flat circular protruding region (320). Protruding region (310) interlocks into a groove region of adaptor (20), while protruding region (320) interlocks into a groove region of slide (40). Such configuration can be applied both to the implantation embodiment and to the translocation embodiment. The bellows can be made integral with the adaptor and the slide by way of a surface seal, in view of the elastic property of silicone as a result of stretching it over the mating surfaces, thus providing the force to keep the surface seal in contact. The internal portion of the nose piece (20) also forms a sealing surface for the bellows. This allows housing portions (51) and (52) to clam shell around the device without requiring a separate sealing.

Turning to the forward portion (41) shown in FIG. 6, together with preventing the bellows (30) from collapsing, such forward portion (41) helps to control velocity of the fluid inside the bellows (30), thus significantly decreasing unnecessary and potentially damaging movement of the nozzle in the patient's eye or subretinal space. In particular, the bellows (30) folds during compression. Such folding reduces the internal length of the bellows (30), thus reducing the space between such internal length and forward portion (41). Such reduction of space due to compression provides acceleration of microfluidic material and/or air inside the chambers of the bellows (30). Such acceleration continues as each chamber of the bellows (30) collapses. The rate of acceleration can be controlled in a design stage by selection of the length of forward portion (41) and by selection of the internal length of bellows (30), and can be controlled, during surgery, by the speed at which engagement member (60) is moved from position B to position C, see FIG. 4. Such considerations apply with reference to the implantation embodiment, the translocation embodiment, and the later discussed preparation embodiment of the present disclosure.

As mentioned in the paragraphs above, while there is, generally speaking, no need to provide an implantation device with an external suctioning ability, such feature can be present in translocation devices. A possible way to structurally design implantation devices and translocation devices in accordance with the present disclosure is that of providing both of them with a suction path and then providing a structural arrangement in the implantation devices to block such path. For example, with reference to FIG. 6 discussed above, where a cross section of a translocation device is shown, fluidic communication between sections (42) and (43) is present. On the other hand, for the implantation device, a configuration like the one shown in FIG. 6B can be provided, where region (44) fluidically disconnects channel (142) from channel (143). Therefore, with implantation devices like the one shown in FIG. 6B, suctioning and negative pressure, if needed, will be exerted through the bellows.

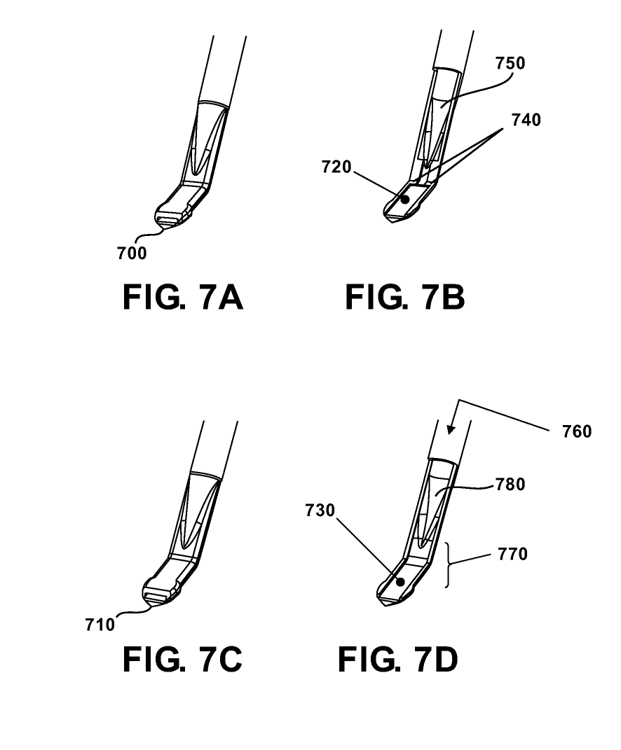

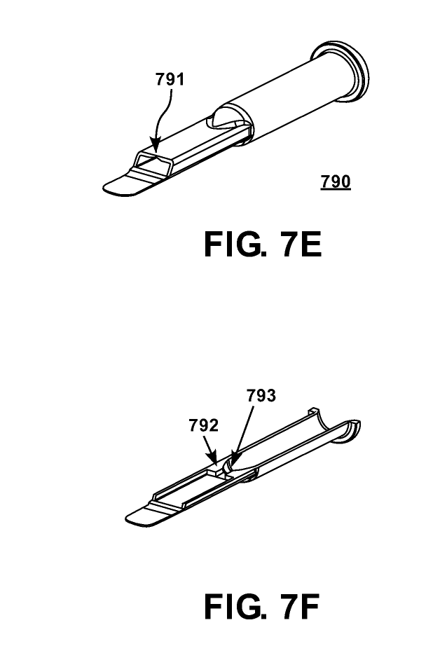

In accordance with aspect further embodiment of the translocation device, the nozzle (10) can be provided with a blocking arrangement to limit the path of the intact cell layers/tissue suctioned into the device. A detailed description of the blocking arrangement is shown in FIGS. 7A-7F.

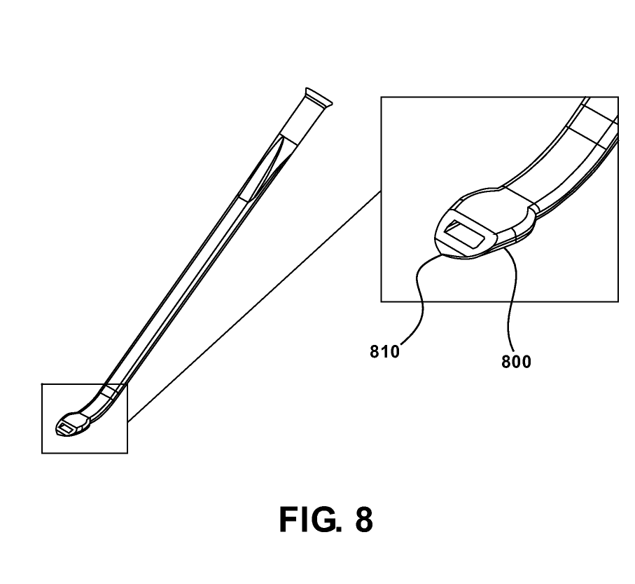

In particular, FIGS. 7A and 7B show a perspective view and a partial sectional view of a nozzle for a translocation device, while FIGS. 7C and 7D show a perspective view and a partial sectional view of a nozzle for an implantation device. Both nozzles have a snake-like head tip (700), (710) located at the distal end of the nozzle, which will be later discussed in detail with reference to FIG. 8.

With continued reference to FIGS. 7A-7D, a tissue chamber (720, 730) is present both in the nozzle for the translocation device and the nozzle for the implantation device. However, differently from the implantation nozzle of FIGS. 7C-7D, the translocation nozzle of FIGS. 7A-7B shows a stop portion (740) to prevent front loaded tissue collected in chamber (720) from entering channel (750), while still keeping tissue chamber (720) and channel (750) in fluidic communication. On the other hand, in the implantation nozzle of FIGS. 7C-7D, where suctioning of cells is not provided for, and where the cells are back loaded from the rear end (760), there is no need of a stop in the bridging region (770) which connects channel (780) with tissue chamber (730).

The stop or bottleneck arrangement or portion (740) prevents the intact cell layers/tissue that is drawn into it from being suctioned too far into the nozzle. A first reason for that is that suctioning the material too far into the nozzle would require higher pressures to be generated in order to express the material out of the nozzle. A second reason is that provision of the stop/bottleneck portion provides for a predetermined position at which the material will be located, thus also allowing the expressing pressure/force to be predetermined. A third reason is that the provision of the stop/bottleneck portion will hold the autologous intact retinal cell layer specimen as close to the distal end of the nozzle to prevent any unnecessary length of travel which could cause distortion or damage to the material by limiting the distance of the material intake into the nozzle and its expression from the nozzle.

A stop portion can also be provided in the intracorneal nozzle already described with reference to FIGS. 5A-5D. In particular, FIGS. 7E-7F show a perspective view and a partial sectional view of an embodiment of an intracorneal nozzle (790). Together with a notch or groove (791) similar to the one described in FIG. 5D, the intracorneal nozzle also comprises a stopping arrangement (792, 793) similar to the one discussed in FIG. 7B. See also FIG. 5C.

As already mentioned above, in accordance with a further embodiment of the present disclosure, the nozzle can have a snake-like head tip, as shown in FIG. 8.

According to such embodiment, the head (800) acts as a lead-in for pushing the nozzle through the retinal incision (retinotomy). Moreover, as the head (800) is pushed further through the retinotomy, it stretches the retinotomy, thus allowing for a larger size nozzle to gently enter the subretinal space through a smaller size retinotomy and the distal end of the nozzle. Still further, upon retraction of the distal end of the nozzle from the retinotomy, the surrounding tissue is expected to return to its previous size due to its elastic properties.

More particularly, the lip (810) is going to enable the surgeon to hold the autologous specimen up against the nozzle and strip the surface of the retina prior to or as the specimen is being suctioned into the nozzle.

The person skilled in the art will understand, upon reading of the present disclosure, that the snake head shape enables translocation, since it is intended to go into the subretinal space. In particular, the nozzle can gently stretch a retinotomy with reduced cutting and essentially atraumatically, by lifting the flap to get into the subretinal space. Moreover, the intact retinal cell layers and/or tissue can be held in their native planar configuration and polarity so that the specimen can be gently placed into the nozzle through suction.

As already previously mentioned, when the material in the nozzle (10) is administered to the patient, the functioning of the translocation device will be identical to the functioning of the implantation device. In other words, the finger-actuated engagement member (60) (see FIG. 3) will be pushed in a B-to-C direction (see FIG. 4) thus compressing the bellows (30) (see FIG. 6) and expressing the fluid and the tissue and/or intact cell layers into the subretinal space.

With continued reference to the translocation embodiment and to FIG. 6, the person skilled in the art will appreciate the dual function of the hollow slide (40). During a capturing condition, there is slide movement away from the nozzle (10) and negative hydraulic pressure is transmitted to the nozzle (10) through the presence of the communicating hollow regions (42), (43) inside the slide (40). On the other hand, during a delivery condition, there is slide movement towards the nozzle (10), but no transmission of negative pressure through the hollow channel.

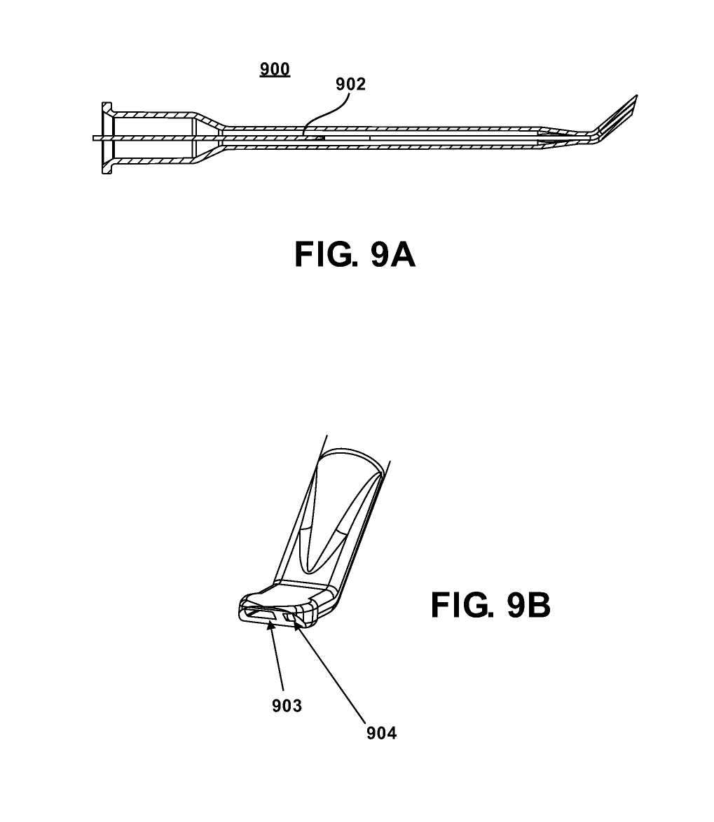

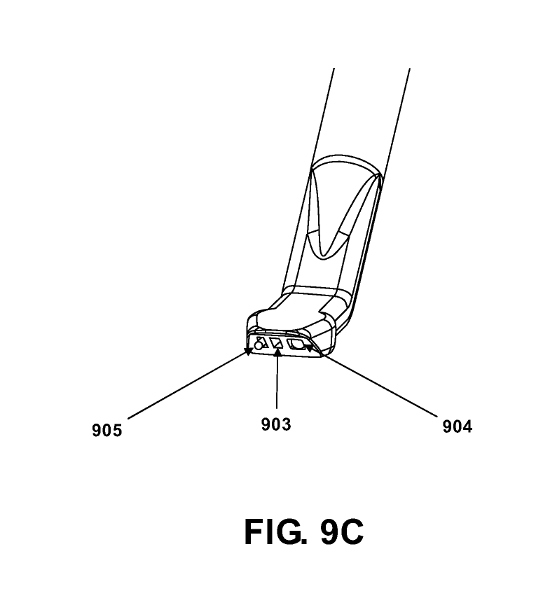

A fourth embodiment of the present disclosure also provides for a preparation device having a nozzle (900) shaped as shown in the sectional view of FIG. 9A and the partial perspective view of FIG. 9B, which is used for removal of debris and other undesirable materials through suction. In particular, the preparation nozzle comprises a fiber optic lumen (902). By way of such device, a surgeon will be able to observe (by way of the fiber optic lumen) the careful removal of all undesirable dead intact cell layers, blood, debris, blood vessels, etc from the subretinal space.

FIG. 9B shows a partial perspective view of the distal end of the nozzle (900) of FIG. 9A, where a perfusion/suctioning tip (903) and lumen (904) are shown.

FIG. 9C shows a further embodiment of a nozzle for a preparation device, where a further opening for a bi-polar cauterization device (905) is also present. The cauterization arrangement can be used by the surgeon to stop bleeding by means of cauterizing tissue/blood vessels in the sub retinal space.

The preparation device has substantially the same functions of the previously described translocation device. However, in its intended use, the preparation device will not express any materials. On the other hand, using the positive pressure of the bellows, the preparation device will perfuse microfluids and avoid damage to the delicate function of cells, vessel walls, blood vessels, etc, in the subretinal space. Moreover, similarly to what already described with reference to the translocation device, using the negative pressure of the bellows, the preparation device will suction undesirable debris and/or material through a fluid path with an external vacuum, as already shown in FIGS. 6 and 6A with reference to the translocation device. It should also be noted that the bellows can be re-filled with microfluids by negative backward motion of an actuator button (see, for example, element (60) shown in FIG. 3) to draw in sterile balance saline solution from the patient's own eye during the surgical preparation procedure.

According to a first example of this embodiment, the preparation device is not intended to have its own mechanism to generate vacuum. As shown in FIG. 9A, the nozzle (900) of the preparation device comprises a fiber optic lumen (902) to allow viewing of material as it is suctioned into the nozzle (900). The fiber optic lumen (902) can be integrated into the nozzle (900) and can be attached to a digital viewing screen (not shown) or directly to an operating microscope (not shown) in order for the surgeon to be able to operate in the subretinal space while having a direct view of the subretinal space and other fragile retinal layers that would otherwise be impossible to view with the naked eye or through a standard operating microscope. Vacuum can be generated through an external vacuum source. In the diagram of FIG. 9A, lumen (902) is shown hatched up to a certain distance to take into account bending on the lumen (902) inside the nozzle (900).

According to a second example of this embodiment, the preparation device also comprises a bellows, similarly to what shown in FIGS. 6 and 6A. As already mentioned above, presence of the bellows allows movement of undesirable material into a better position for suction removal by way of perfusion of microfluids.

In addition to applications in the field of retinal surgery, the preparation device can have applications in brain, inner ear, spinal cord surgery, and other areas in the human body, where direct observation is required, to avoid damage to the central nervous system, peripheral nervous system, sensory tissues and cell structures.

A further embodiment of the present disclosure provides for a nozzle cover also acting as a cartridge, as shown in the cross sectional views of FIGS. 10, 10A and 10B of the present application. As shown in FIGS. 10 and 10A, a cover (400) is provided, having a substantially cylindrical shape, thus adapting the cover (400) to be used with any of the nozzle shapes shown in the present disclosure.

A first use of the cover (400) is that of protecting the nozzles from damage and to allow transportation and storage of the device. Protection of the nozzle is especially important in the implantation embodiments, where intact cell layers, to be later implanted, are present and have to be protected from damage or disruption.

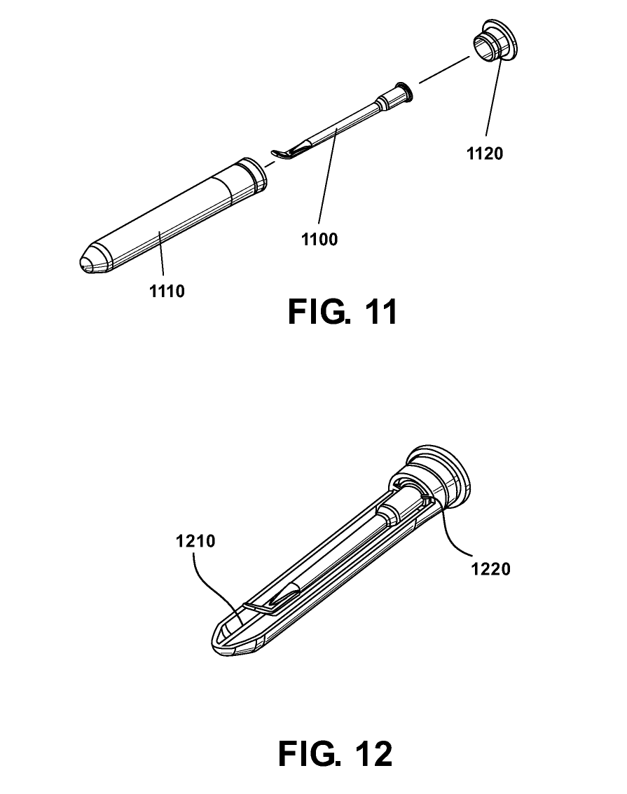

An additional use of the cover (400) is to enable independent, multiple covers to be used as cartridges and accompany all of the embodiments of the devices according to the present disclosure in order to provide for multiple implants in pre-loaded implantation nozzles and other nozzles that may need to be replaced due to conditions such as damage or intentional mishandling. Therefore, embodiments of the present disclosure can be provided, where one or more covers are independently packaged to allow for more than one implant to be used with a single hand piece and for replacement purposes, should the primary nozzle become damaged or otherwise rendered unusable. These embodiments will later be discussed more in detail, with reference to FIGS. 11 and 12.

As shown in FIGS. 10 and 10A, the cover (400) is connected with the nose piece or adaptor (20) through a snap-fit sealing connection. In accordance with the example shown in FIGS. 10 and 10A, such sealing connection is obtained by configuring the nose piece or adaptor (20) to exhibit a bulge in correspondence of region (201), which is compressed as soon as cover (400) is inserted around the nozzle (405), thus forming the connection. Further, as shown in the example of FIG. 10A, the external wall (410) abuts on shoulder portion (500) of the rest of the device.

The snap-fit connection discussed above creates a water-tight seal. One of the consequences of this kind of seal is that preservation and storage media can be contained around the nozzle (405). Moreover, the seal allows integration between the cover and the hand piece, thus creating a one-piece, single shipment capability to its destination.

The material of which the cover is made can be transparent or clear, so that the storage media fluid level can be readily appreciated, thus allowing the cover to be removed without damaging the device or live cells within.

FIG. 10B shows a further embodiment of the cover according to the present disclosure. Cover (420) of FIG. 10B comprises a stop (425) for a nozzle (430). Cover (420) also comprises supporting ribs (435) and (440). Supporting rib (435) is adapted to keep the rear portion and the main body of the nozzle (430) in place inside the cover (420), while supporting rib (440) is adapted to keep the distal portion and the tip of nozzle (430) in place. If desired, cover (420) can also comprise a supporting wall (445) to block tissue from escaping from the nozzle (430). Cover (420) further comprises a chamber (450), where storage and preservation media can be included.

Reference will now be made to embodiments where one or more covers are independently packaged as cartridges to allow more than one implant to be used with a single hand piece and for replacement purposes, should the primary nozzle become damaged or otherwise rendered unusable. Examples of these embodiments are shown in the following FIGS. 11 and 12. FIG. 11 shows an exploded perspective view, where a nozzle (1100) is inserted into a cover (1110) and locked by a cap (1120). To better support the nozzle inside the cover, supporting ribs can be provided, as shown in FIG. 12, which shows a cutout view of the embodiment of FIG. 11. In particular, FIG. 12 shows supporting ribs (1210) and (1220).

Example surgical procedure protocols making use of the devices discussed above will now be described. A first example relates to a translocation surgical procedure protocol. A second example relates to a retinal implantation surgical procedure protocol. A third example relates to a corneal transplantation surgical procedure protocol.

### FIRST EXAMPLE

### Translocation Surgical Procedure Protocol

### 1) Description

In neovascular Age-related Macular Degeneration (NAMD) patients, the neovascular network or membrane originates from the choroid which grows through Bruch's membrane and grows either above or below the RPE (retinal pigment epithelium) layer. Translocation involves the surgical removal of the neovascular membrane followed by the autologous translocation of full thickness retina specimen excised from the mid-periphery of the patient's own eye.

### 2) Surgical Kit for Translocation Procedure

a) Preparation device in accordance with the embodiments described above (see, e.g., the nozzle shown in FIGS. 9A-9C). In patients with NAMD, there are remnants of dead and/or dying RPE cells and other debris under the macula. The preparation device will be inserted in a retinotomy to suction and clear the debris field under the macula prior to translocation of autologous retinal cell layers in order to prevent inflammation and contamination of the new cell layers and adjacent RPE cells. As discussed with reference to FIGS. 9A-9C, the preparation device has microfiber optic viewing capability along with perfusion and suction in order to prepare the retinal area for the best possible vision recovery in patients.
b) Translocation device in accordance with the embodiments described above (see, e.g., FIGS. 6, 6A, 7A, 7B and 8). Subsequent to the removal of the neovascular membrane, dead and dying cells, blood and other debris in patients, the translocation device gently suction loads an excised autologous full-thickness retina specimen (intact choroid, Bruch's membrane and RPE). Then, the translocation device relocates the specimen through the retinotomy where the neovascular membrane was removed.
c) 5 mm microvitreoretinal (MVR) blade to extend sclerotomy for translocation.
d) Subretinal V forceps.
e) Replacement translocation nozzle cartridges in accordance with the embodiments described above (see, e.g., FIG. 10B) for backup.

### 3) Procedure

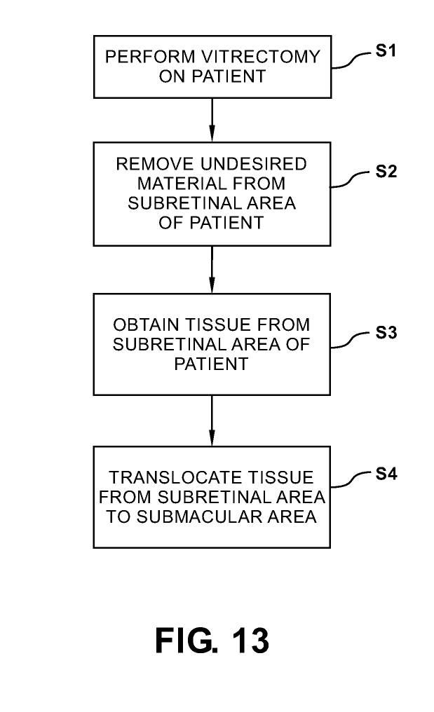

a) Standard pars plana vitrectomy (PPV) with complete posterior hyaloid dissection. See also step S1 of FIG. 13.
b) If choroidal neovascularization (CNV) present, remove via standard techniques (e.g., temporal incision).
c) Removal of subretinal hemorrhage, cells and/or other debris in the submacular area with the preparation device. The preparation device will loosen and remove dead cells, hemorrhage, debris, provide perfusion and real-time digital visualization of the subretinal area during the procedure. See also step S2 of FIG. 13.
d) Identify site of retina to be translocated. For example, an inferior site can be chosen, as it affects only the superior visual field.
e) The following techniques can be applied to optimally obtain the translocation tissue (see also step S3 of FIG. 13):
e1) Cauterization of the retina and choroid around a 2.4 by 4.0 mm section of retina. Create an incision with an MVR blade or vertical scissors. Extend the sclerotomy for the insertion of the translocation device followed by removal of the superficial retina with a lighted pick, modified Charles needle, or forceps prior to or as the autologous specimen (e.g., choroid, Bruch's membrane and/or RPE layer) in its native planar configuration and polarity is suctioned into the translocation device.
e2) Detachment of the area of retina overlying the autologous specimen to be translocated with a macular translocation needle. Cauterization and incision of the retina to allow access to the underlying tissue and cauterization of the 2.4 by 4.0 mm section of the autologous specimen. Extend the sclerotomy to 5 mm. Removal as above with the translocation device.
f) Move the translocation device, loaded with the autologous specimen, and insert the translocation device into the submacular area gently stretching the retinotomy with the translocation device followed by the expression of the autologous specimen into the submacular location underlying the fovea. Gently remove the device to allow the retina to close and keep the tissue in place. See also step S4 of FIG. 13.
g) Laser around the retinotomy at the translocation site and ensure that there is air-fluid exchange. Alternatively, use perfluoro-n-octane (PFO) to flatten the macula and ensure that no submacular fluid remains. Perform laser to the retinotomy at the translocation site and then perform an air-fluid exchange filling the eye with a tamponade of gas or silicone oil.
h) Perform a standard vitrectomy wound closure.

### SECOND EXAMPLE

### Retinal Implantation Surgical Procedure Protocol

### 1) Description

In patients with Atrophic Age-related Macular Degeneration (AAMD), the retinal implantation surgical procedure will implant immature, intact RPE and neurosensory retinal cell layers in the subretinal space.

### 2) Surgical Kit for Retinal Implantation Procedure

a) Preparation device in accordance with the embodiments described above (see, e.g., the nozzle shown in FIGS. 9A-9C). In patients with AAMD, there are remnants of dead and/or dying RPE cells and other debris under the macula. The preparation device will be inserted in a retinotomy to suction and clear the debris field prior to the retinal implantation procedure in order to prevent inflammation and contamination of the new cell layers and adjacent RPE cells. As discussed with reference to FIGS. 9A-9C, the preparation device has microfiber optic viewing capability along with perfusion and suction in order to prepare the retinal area for the best possible vision recovery in patients.
b) Implantation device in accordance with the embodiments described above (see, e.g., FIGS. 1-4). The implantation device will safely and atraumatically implant immature, intact RPE and neurosensory cell layers (2.4×4 mm in size) in the required location of the sub-retinal space.
c) 5 mm MVR blade to extend sclerotomy for retinal implantation.
d) Replacement, pre-loaded implantation nozzle cartridges in accordance with the embodiments described above (see, e.g., FIG. 10B) for multiple implants and backup.

### 3) Procedure

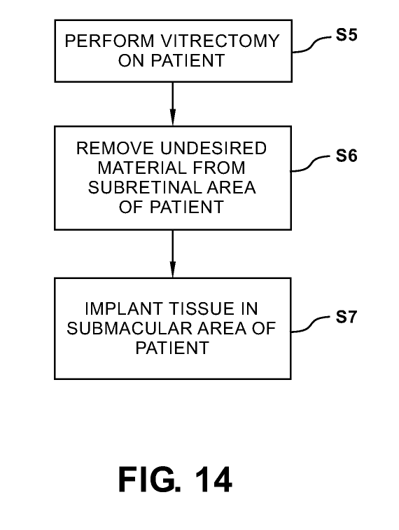

a) Standard PPV with complete posterior hyaloid dissection. See also step S5 of FIG. 14.
b) Create a 20 gauge retinotomy at the submacular implantation site.
c) Removal of subretinal dead and dying cells and other debris with contagions and/or toxins in the submacular area using the preparation device to provide perfusion and allow for real-time digital visualization of the subretinal area during the procedure. See also step S6 of FIG. 14.
d) Extend the sclerotomy to 5 mm.
e) Insert the implantation device through the sclerotomy into the subretinal space by gently stretching the retinotomy with the implantation device followed by the expression of intact retinal cell layers, 2.4×4.0 mm, in the desired location of the submacular area. See also step S7 of FIG. 14.
f) If multiple implants are required, replace the used implantation nozzle with a new preloaded nozzle cartridge and repeat the same procedure as described in e).
g) Laser around the retinotomy at the retinal implantation site and ensure that there is air-fluid exchange. Alternatively, use PFO to flatten the macula and ensure that no submacular fluid remains.
h) Perform a standard vitrectomy wound closure.

### THIRD EXAMPLE

### Corneal Transplantation Surgical Procedure Protocol

### 1) Description

Corneal decompensation and poor vision can result from a variety of diseases, trauma, and chemical damage. The decompensation is manifested by corneal edema (swelling) with epithelial edema. The edema is actually due to dysfunction of the endothelial cells on the posterior surface of the cornea that remove fluid from the cornea and maintain its clarity. In the past a penetrating keratoplasty has been performed, however more recently it was discovered that the endothelial layer and Descemet's membrane can be transplanted without the entire cornea and the transplanted cells clear the cornea (DSEK).

### 2) Surgical Kit for Corneal Endothelial Transplantation

a) Corneal transplantation device in accordance with the embodiments described above (see, e.g., the device of FIGS. 1-4 with the nozzle shown in FIGS. 5A-5D). The device is designed to contain the Descemet's membrane and endothelial layer from a donor cornea stored in Optisol®.
b) Corneal transplantation forceps designed to assist in pulling the transplant into the anterior chamber (AC); through a fluid connection, air or hyaluronic acid can be injected into the AC to hold the transplant in place.

### 3) Procedure

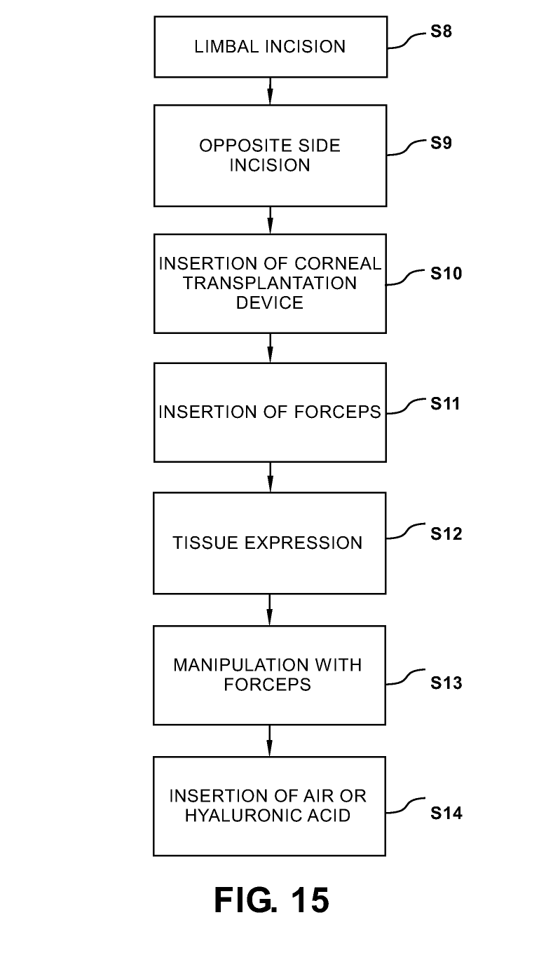

a) Removal of the cornea endothelium and Descemet's membrane.
b) Limbal incision extension to 5 mm if not already that size (see step S8 of FIG. 15). 20 gauge incision for the forceps, to be made 180 degrees from the 5 mm incision (step S9 of FIG. 15).
c) Inserting the corneal transplantation device into the 5 mm incision with the glide to protect the iris, cover the pupil, and provide a stable surface for the transplantation (step S10 of FIG. 15).
d) Inserting forceps through the opposite side incision (step S11), expressing the tissue from the device (step S12), and manipulating with the forceps (step S13). Air or hyaluronic acid is injected (step S14) through the injection port of the device in the forceps to keep the transplantation tissue in a proper location.

The foregoing detailed description of exemplary and preferred embodiments is presented for purposes of illustration and disclosure in accordance with the requirements of the law. It is not intended to be exhaustive nor to limit the invention to the precise form or forms described, but only to enable others skilled in the art to understand how the invention may be suited for a particular use or implementation. The possibility of modifications and variations will be apparent to practitioners skilled in the art. No limitation is intended by the description of exemplary embodiments which may have included tolerances, feature dimensions, specific operating conditions, engineering specifications, or the like, and which may vary between implementations or with changes to the state of the art, and no limitation should be implied therefrom.

This disclosure has been made with respect to the current state of the art, but also contemplates advancements and that adaptations in the future may take into consideration of those advancements, namely in accordance with the then current state of the art. It is intended that the scope of the invention be defined by the claims as written and equivalents as applicable. Reference to a claim element in the singular is not intended to mean “one and only one” unless explicitly so stated.

Moreover, no element, component, nor method or process step in this disclosure is intended to be dedicated to the public regardless of whether the element, component, or step is explicitly recited in the Claims. No claim element herein is to be construed under the provisions of 35 U.S.C. Sec. 112, sixth paragraph, unless the element is expressly recited using the phrase “means for . . . ” and no method or process step herein is to be construed under those provisions unless the step, or steps, are expressly recited using the phrase “comprising step(s) for . . . ”

1. A device adapted to implant fluidic material in a patient's eye, comprising:
a nozzle; and
a housing connected with the nozzle, the nozzle comprising a slide; a holding region, a glide located downstream of a tip region of the nozzle, and a cover
a cap configured to lock the nozzle inside the cover;
wherein:
the slide is adapted to slide towards the nozzle or away from the nozzle/

2. (canceled)

3. The device of claim 77, wherein the bellows is integrally connected with the slide along a first end region of the bellows.

4. The device of claim 1, wherein the nozzle contains the fluidic material to be implanted.

5. The device of claim 77, further comprising an adaptor located between the nozzle and the bellows.

6. The device of claim 77, wherein the adaptor is integrally connected with the bellows along a second region of the bellows.

7. The device of claim 1, further comprising a movement element integral with the slide, the movement element being adapted to be hand-operated to cause sliding of the slide towards or away from the nozzle.

8. The device of claim 7, wherein the housing comprises a cut-out portion, the movement element protruding from the cut-out portion.

9. The device of claim 8, wherein the movement element and the cut-out portion are configured as a locking arrangement, the locking arrangement adapted to assume a blocking condition where movement of the slide is blocked and a sliding condition where movement of the slide is allowed.

10. The device of claim 9, wherein the cut-out portion is substantially L-shaped and wherein the movement element is adapted to move along a first direction of the L-shaped cut-out portion to block or unblock the slide and is adapted to move along a second direction of the L-shaped cutout portion to allow sliding of the slide.

11. The device of claim 10, wherein the movement of the movement element along the first direction is a rotational movement and the movement of the movement element along the second direction is a translational movement.

12. The device of claim 1, wherein the nozzle comprises a bevel region.

13. The device of claim 12, wherein the bevel region has an inclination between about 45 degrees and about 60 degrees.

14. The device of claim 77, further comprising a forward portion attached to the slide, the forward portion located inside the bellows.

15. (canceled)

16. (canceled)

17. The device of claim 1, wherein the tip region of the nozzle is tapered.

18. The device of claim 1, wherein the holding region comprises a groove.

19. The device of claim 1, wherein the holding region is a hollow holding region.

20. The device of claim 1, wherein the nozzle comprises a body region fluidically connected with the holding region and wherein the holding region comprises a stopping arrangement to prevent material inside the holding region from leaving the holding region.

21. The device of claim 77, wherein the slide has a hollow interior thus establishing a fluid path from the nozzle to the slide through the bellows, and through the slide.

22. The device of claim 21, wherein a combination of the fluid path together with a) sliding away of the slide from the nozzle or b) suctioning of air or fluid from the slide generates negative pressure forming a suctioning effect.

23. The device of claim 1, wherein the slide has a hollow interior portion fluidically connected with the nozzle.

24. The device of claim 1, wherein the slide has a hollow interior portion fluidically disconnected from the nozzle.

25. The device of claim 14, wherein the forward portion and the slide comprise hollow channels in fluidic communication.

26. The device of claim 25, wherein the forward portion hollow channel and the slide hollow channel have different diameters.

27. The device of claim 14, wherein the forward portion and the slide comprise fluidically separate hollow channels.

28. The device of claim 9, wherein, in the sliding condition, the slide moves towards the nozzle or away from the nozzle, and wherein movement of the slide towards the nozzle is adapted to expel the fluidic material from the nozzle, and movement of the slide away from the nozzle is adapted to capture material and/or fluid inside the nozzle and/or the bellows.

29. The device of claim 28, wherein the capture of the fluidic material inside the nozzle is performed through a suctioning arrangement.

30. The device of claim 29, wherein the fluidic material expelled from the nozzle is the same material previously suctioned inside the nozzle.

31. The device of claim 77, wherein the bellows comprises a first end and the slide comprises a groove region, the bellows being connected with the slide through interlocking of the first end into the groove region.

32. The device of claim 31, wherein the first end comprises a flat protruding region.

33. The device of claim 32, wherein the flat protruding region is a flat circular protruding region.

34. The device of claim 5, wherein the bellows comprises a second end and the adaptor comprises a groove region, the bellows being connected with the adaptor through interlocking of the second end into the groove region.

35. The device of claim 34, wherein the second end comprises a flat protruding region.

36. The device of claim 35, wherein the flat protruding region is a flat circular protruding region.

37. The device of claim 14, wherein a combination between the forward portion and the bellows acts as a fluid velocity control arrangement during sliding of the slide towards the nozzle.

38. The device of claim 1, wherein the nozzle comprises a snake head shaped tip.

39. The device of claim 1, wherein the nozzle comprises a stop or bottleneck arrangement.

40. The device of claim 39, wherein the stop or bottleneck arrangement is configured to control positioning of suctioned material into the nozzle.

41. A nozzle for intracorneal implantation surgical procedures, comprising: a body region;
a tip region;
a holding region between the body region and the tip region, the holding region defining a channel adapted to be filled with fluid and contain corneal cell layers for implantation; and
a lens glide located after the tip region.

42. The nozzle of claim 41, wherein the tip region is a tapered tip region.

43. The nozzle of claim 41, wherein the holding region comprises a groove, the groove being adapted to allow the corneal cell layers to be grasped during operation.

44. The nozzle of claim 43, wherein the groove is located in the upper center of the holding region.

45. The nozzle of claim 41, wherein the holding region comprises a stopping arrangement to prevent the corneal cell layers from leaving the holding region.

46. The nozzle of claim 41, wherein the tip region is a snake head shaped tip region.

47. A nozzle for surgical procedures, the nozzle adapted to be connected to a surgical procedure preparation device, the nozzle comprising:
a first lumen, to allow vision of operation of the surgical procedure preparation device on a patient's body, and
a nozzle tip, the nozzle tip comprising
i) a first opening into which material is adapted to be suctioned from the patient's body or from which material is adapted to be injected into the patient's body, and
ii) a second opening where a distal end of the lumen is located.

48. The nozzle of claim 47, further comprising a second lumen, to host a cauterization arrangement.

49. A preparation device, comprising the nozzle of claim 47.

50. The preparation device of claim 49, further comprising a housing, connected with the nozzle, the housing comprising a bellows, wherein the bellows is adapted to undergo compression or decompression.

51. The preparation device of claim 50, wherein the decompression of the bellows is for perfusing additional material into the patient's body.

52. The preparation device of claim 50, wherein the bellows has a hollow interior thus establishing a suctioning fluid path from the nozzle through the bellows to suction the material through generation of negative pressure.

53-76. (canceled)

77. The device of claim 1, wherein the housing further comprises a bellows located between the nozzle and the slide, wherein the bellows is configured to undergo compression and decompression.
